(19) United States
(12) Patent Application Publication
(10) Pub. No.: US 2012/0123241 A1
(43) Pub. Date: May 17, 2012

(54) EXTERNAL MEDICAL DEVICE WARNING OTHER MEDICAL DEVICE OF IMPENDING ADMINISTRATION OF TREATMENT

(75) Inventors: Michael C. Stilley, Lino Lakes, MN (US); John Robert Knapinski, Kirkland, WA (US); Robert Garland Walker, Seattle, WA (US); David J. Jorgenson, Bloomington, MN (US)

(73) Assignee: Physio-Control, Inc., Redmond, WA (US)

(21) Appl. No.: 13/359,415

(22) Filed: Jan. 26, 2012

Related U.S. Application Data
(63) Continuation-in-part of application No. 12/258,872, filed on Oct. 27, 2008.
(60) Provisional application No. 61/543,767, filed on Oct. 5, 2011.

(51) Int. Cl. A61B 5/055; A61N 5/10; A61N 7/00; G08B 21/02; A61N 1/00; A61H 31/00

(52) U.S. Cl. 600/410; 607/5; 601/41; 601/2; 340/573.1; 378/65

(57) ABSTRACT

In one embodiment, an external first medical device includes a treatment module for administering a first treatment. The first medical device also includes a warning module for transmitting, before the first treatment is administered, a warning about the impending administration of the first treatment. The warning is suitable for receipt by a second medical device that could be attached to the patient unbeknownst to the first device, and which could be impacted by the first treatment. As such, the second medical device is given the opportunity to take a defensive action.

### CROSS REFERENCE TO RELATED PATENT APPLICATIONS

This patent application claims priority from U.S. Provisional Patent Application Ser. No. 61/543,767, filed on Oct. 5, 2011, the disclosure of which is hereby incorporated by reference for all purposes, Attorney Docket No. C00001411.S0.

This patent application is a continuation-in-part of co-pending U.S. patent application Ser. No. 12/258,872, filed on Oct. 27, 2008, entitled DEFIBRILLATOR WITH IMPLANTABLE MEDICAL DEVICE DETECTION, commonly assigned herewith, Attorney Docket No. PB0010034.02.

This patent application may be found to be related to U.S. patent application Ser. No. [SER_NO_OF_C3074-USN1], titled IMPLANTABLE MEDICAL DEVICE WARNING OTHER MEDICAL DEVICE OF IMPENDING ADMINISTRATION OF TREATMENT, filed on the same day as the present application, in the name of the same inventors, and commonly assigned as of the date this document is initially filed in the Patent Office, and with Attorney Docket No C00003074.USN1.

This patent application may be found to be related to U.S. patent application Ser. No. [SER_NO_OF_C1411-USN2], titled EXTERNAL MEDICAL DEVICE REACTING TO WARNING FROM OTHER MEDICAL DEVICE ABOUT IMPENDING INDEPENDENT ADMINISTRATION OF TREATMENT, filed on the same day as the present application, in the name of the same inventors, and commonly assigned as of the date this document is initially filed in the Patent Office, and with Attorney Docket No C00001411.USN2.

This patent application may be found to be related to U.S. patent application Ser. No. [SER_NO_OF_C3080-USN1], titled IMPLANTABLE MEDICAL DEVICE REACTING TO WARNING FROM OTHER MEDICAL DEVICE ABOUT IMPENDING INDEPENDENT ADMINISTRATION OF TREATMENT, filed on the same day as the present application, in the name of the same inventors, and commonly assigned as of the date this document is initially filed in the Patent Office, and with Attorney Docket No C00003080.USN1.

### FIELD

This invention generally relates to medical devices.

### BACKGROUND

External devices and implantable devices are examples of medical devices.

For purposes of this document, “external medical device” means a device that is applied to the patient primarily externally, often by attaching in some way. Attaching can be in direct contact with their skin, or through their clothing. Sometimes it can penetrate the skin, for example for intravenous administration of a drug. Examples of external medical devices are given later in this document.

For purposes of this document, “implantable medical device” means a device that has been implanted within a patient, wholly or in part. Implanting is not temporary in that at least a component of the implantable medical device remains under the patient's skin for some time longer than, for example, seven days. An implantable device is necessarily attached to the patient while it is implanted! Examples of implantable medical devices are given later in this document.

Medical devices are becoming increasingly sophisticated. They administer treatment. They monitor and measure physiological parameters of patients. Sometimes they do both. Sometimes the parameter they measure determines what treatment to administer, and when. An example is now described.

FIG. 1 shows a first medical device MEDICAL DEVICE A 141 attached to patient 122. Medical device 141 can be external or implantable. Medical device 141 includes a treatment module 148 that is capable of delivering a treatment 149 to patient 122. Medical device 141 optionally also includes a sensing module 146, for sensing a physiological parameter of patient 122. Medical device 141 may optionally include a controller 152 for controlling treatment module 148, for sensing module 146 if provided, and perhaps for other internal operations.

FIG. 1 shows a second medical device MEDICAL DEVICE B 162 attached to patient 122. Medical device 162 can be external or implantable. Medical device 162 includes an operation module 166 for operating on patient 122. Operation module 166 can include either one or both of a sensing module 168-A, a treatment module 168-B, and so on. Medical device 162 may optionally include a controller 172 for controlling operation module 166, and perhaps for other internal operations.

Sometimes multiple devices are attached to the same patient, as is shown in the example of FIG. 1. In particular, both first medical device 141 and second medical device 162 are attached to the same patient 122, concurrently.

A risk can occur when two or more medical devices are tending to the same patient at the same time. More specifically, as seen in the example of FIG. 1, treatment 149 by first medical device 141 could affect at least a quality of the later operation of second medical device 162. Affecting could be in a number of ways, depending on the nature of treatment 149 and of operation module 166. For example, if treatment 149 is an electrical discharge, such as from a defibrillator, the discharge might affect temporarily or damage permanently components of operation module 166, thus affecting the quality of their later operation. In some particular instance, treatment 149 might prevent sensing module 168-A from operating properly, such as when sensing module 168-A is receiving ECG readings.

In some instances, the risk is ameliorated by circumstances. For example, one of the medical devices could have knowledge of the other medical device, to use an anthropomorphic term for the engineering design sense. Knowledge, then, can be by the one device receiving user inputs about the second device, or detecting it, or communicating with it. Examples of such detecting have been described in U.S. Pat. No. 5,951,483, which is hereby incorporated by reference. Examples of such communicating have been described in U.S. Patent Application Pub. Doc. US20060173498, which is hereby incorporated by reference in this document. Knowledge means that internal controls within the medical device are adjusted in certain way to accommodate for the other device being attached to the patient.

Knowledge by the one device of the other is not easy to coordinate or plan for. As such, the risk to second medical device 162 remains, shown in FIG. 1 as an adverse side effect of treatment 149, and more particularly impacting operation module 166.

### BRIEF SUMMARY

The present description gives instances of medical devices, systems, software and methods, the use of which may help overcome problems and limitations of the prior art.

In one embodiment, an external first medical device includes a treatment module for administering a first treatment. The first medical device also includes a warning module for transmitting, before the first treatment is administered, a warning about the impending administration of the first treatment. The warning is suitable for receipt by a second medical device that could be attached to the patient unbeknownst to the first device, and which could be impacted by the first treatment. As such, the second medical device is given the opportunity to take a defensive action.

These and other features and advantages of this description will become more readily apparent from the following Detailed Description, which proceeds with reference to the drawings, in which:

### BRIEF DESCRIPTION OF THE DRAWINGS

FIG. 1 is a diagram of medical devices in the prior art, and further illustrating a problem in the prior art.

FIG. 2A is a diagram illustrating medical devices made according to embodiments, and a further example of their interaction according to embodiments.

FIG. 2B is a diagram of a second medical device of FIG. 2A, further conceptually illustrating a defensive action that it can take according to embodiments.

FIG. 3 shows flowcharts for illustrating methods according to embodiments, and includes indications for additional clarity.

FIG. 4 is a diagram illustrating sample timing of actions of the first medical device of FIG. 2A.

FIG. 5 is a diagram illustrating a sample embodiment of the actions of FIG. 4.

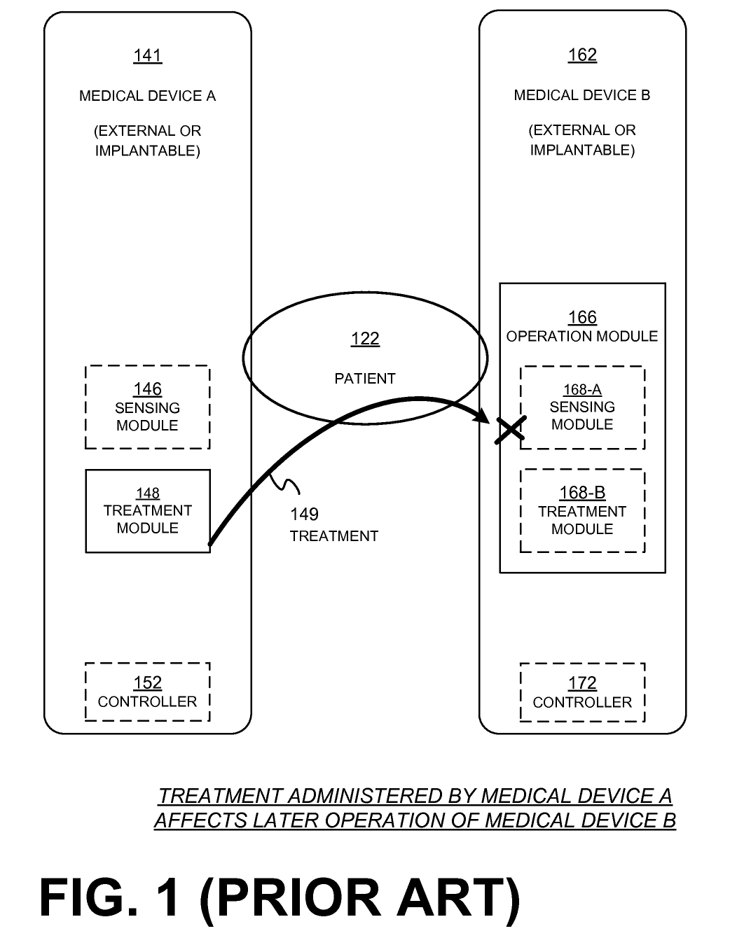

### DETAILED DESCRIPTION

The present description gives instances of medical devices, systems, software and methods, the use of which may help overcome problems and limitations of the prior art. Embodiments are now described in more detail.

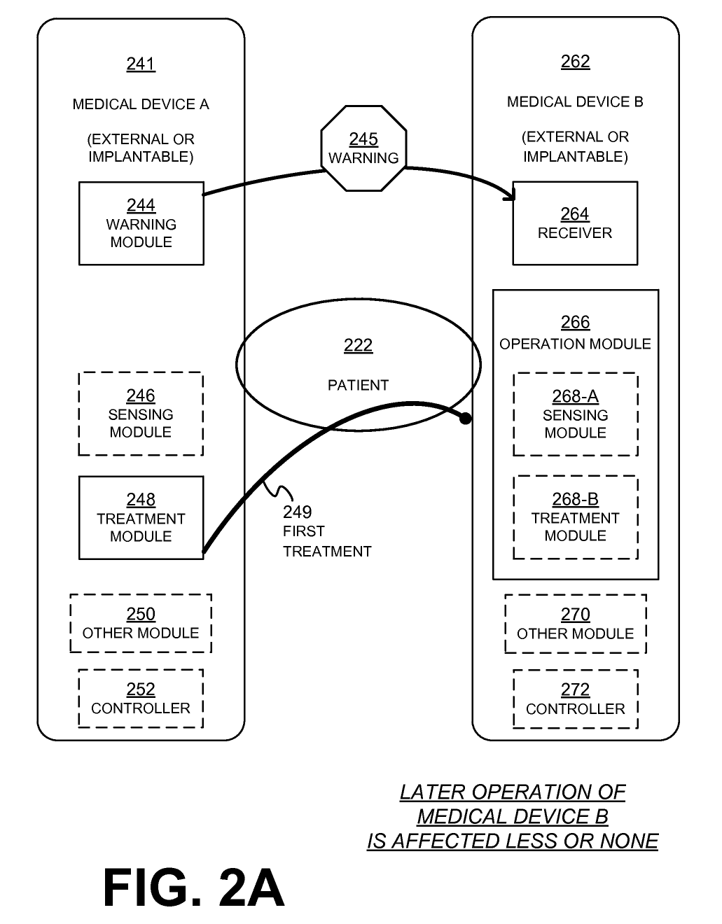

FIG. 2A is a diagram illustrating a first medical device 241 and a second medical device 262, each made according to embodiments. Devices 241, 262 are attached to a patient 222 at the same time. FIG. 2A further shows an example of the interaction between devices 241, 262 according to embodiments. It will be understood, however, that this interaction takes place if at least one of the devices warns, as does device 241, and at least another of the devices reacts to such a warning, as does device 262.

First medical device MEDICAL DEVICE A 241 can be an external or implantable device.

Particular examples of first medical device 241 as an external device include external defibrillators, whether monitor-defibrillators or Automated External Defibrillators (“AED”s), CPR (CardioPulmonary Resuscitation) chest compression devices, devices that can inject drugs intravenously in a rescue scene, before patient 222 is taken to the treatment center, ultrasound machines, devices cooling the patient, for example by injecting a cold fluid, X-Ray machines, MRI machines, and so on.

Particular examples of first medical device 241 as an implantable device include implantable stimulation devices that generate and deliver electrical stimuli to body nerves and tissues for the therapy of various biological disorders. Examples are pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc.

Medical device 241 includes a treatment module 248 that is capable of delivering a first treatment 249 to patient 222. First treatment 249 is in accordance with the nature of device 241.

Medical device 241 moreover includes a warning module 244. Warning module 244 is capable of transmitting, before first treatment 249 is administered, a warning 245 about the impending administration of first treatment 249.

In some embodiments, a preparatory operation for administering first treatment 249 is performed before first treatment 249 is administered. The preparatory operation is performed by any part of device 241, for example a component coupled to, or controlling or affecting treatment module 249. The preparatory operation can also be performed by treatment module 249. In these embodiments, warning 245 is transmitted responsive to the preparatory operation being performed.

Warning 245 is intended to warn second medical device 262, so it could protect itself and/or its operations, as will be understood from this document, in the event medical device 262 is attached to patient 222. Indeed, second medical device 262 could be attached to patient 222 without the knowledge of first medical device 241.

As such, warning 245 is suitable for receipt by second medical device 262, if it is attached to patient 222. This can be accomplished in a number of ways. In some embodiments, warning 245 is transmitted through the body of the patient, and received accordingly by second medical device 262. In some embodiments, warning 245 is transmitted as a sound wave, whether within the audible frequency range or not. In some embodiments, warning 245 is transmitted as a magnetic wave. In some embodiments, warning 245 is transmitted as an electromagnetic wave. The warning could further encode information, as is described later in this document.

Warning 245 is preferably transmitted so that it will be received preferentially by medical device 262, but not too many other medical devices in the vicinity. Otherwise, the situation could become confusing as to which device is intended to receive the warning. Accordingly, in some embodiments, warning 245 is transmitted at a suitably lower power, with the suitability being determined by the expected scenario of detection. In some embodiments, a range can be decided, for example the power is low enough to where the warning is not detectable beyond a distance of 15 feet. In preferred embodiments, where second medical device 262 is attached to the body of the patient the power can be low enough to where the warning is not detectable beyond the patient's body.

Medical device 241 moreover optionally also includes an other module 250, for others of its operations, consistent with its use and function. For example, other module 250 could be a memory, a communications module, a user interface, and so on, as applicable. If other module 250 is a memory, it could store a record about the transmitted warning.

In some embodiments, other module 250 is a receiver that is suitable for receiving an acknowledgement, which has been sent in response to the warning. In other words, it turns out that second medical device 262 is indeed attached to patient 222, has received warning 245, and is acknowledging it. The acknowledgment can be received in a number of ways. In some embodiments, the acknowledgement is received directly from second medical device 262, which has a suitable transmitter, and so on. In other embodiments, the acknowledgement is received from an external network on behalf of second medical device 262. In these other embodiments, second medical device 262 communicates directly with an intermediary device, which transmits the acknowledgement to receiver 250 directly or via a network, and so on. For example, if second medical device 262 is an implantable device, the intermediary device can be a programmer, and so on.

Medical device 241 optionally also includes a sensing module 246, for sensing a physiological parameter of patient 222. The physiological parameter can be any patient parameter including, as an example but not limitation, temperature, ECG, blood-related parameters, and so on.

Medical device 241 may optionally include a controller 252 for controlling treatment module 248, for sensing module 246 if provided, for other module 250 if provided, and perhaps other internal operations.

These features and capabilities are not limiting for device 241. Indeed, device 241 could also include features shown for device 262, and so on. For example, device 241 could further include a receiving module, which could be made similarly or differently from receiver 264 of medical device 262, which is described later in this document. The receiving module of device 241 can be for receiving an acknowledgement in response to the warning, in which case second medical device 262 is indeed attached to patient 222. The acknowledgement can be received directly from medical device 262, or from an external network, on behalf of medical device 262. Moreover, device 241 may include functionality to transmit a confirmation, in response to receiving the acknowledgement. The functionality can be warning module 244, or other module 250, depending on the design.

Second medical device MEDICAL DEVICE B 262 can be an external or implantable device.

Particular examples of second medical device 262 as an external device include external defibrillators, whether monitor-defibrillators or Automated External Defibrillators (“AED”s), CPR (CardioPulmonary Resuscitation) chest compression devices, devices that can inject drugs intravenously in a rescue scene, before patient 222 is taken to the treatment center, ultrasound machines, devices cooling the patient, for example by injecting a cold fluid, X-Ray machines, MRI machines, and so on.

Particular examples of second medical device 262 as an implantable device include implantable stimulation devices that generate and deliver electrical stimuli to body nerves and tissues for the therapy of various biological disorders. Examples are pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc.

Medical device 262 could be attached to patient 222 while having no other knowledge that first medical device 241 is also attached to patient 222. Medical device 262 includes a receiver 264 for receiving warning 245. Receiver 264 is made using a technology in view of how warning 245 is transmitted. From the point of view of medical device 262, warning 245 received by receiver 264 is about an impending administration to patient 222 of first treatment 249 by a different device, in this case device 241.

Medical device 262 includes an operation module 266 for operating on patient 222. Operation module 266 can include either one or both of a sensing module 268-A, a treatment module 268-B, and so on. Sensing module 268-A could be for sensing the same parameters as module 246. Accordingly, the operation of module 266 can be monitoring a physiological parameter of patient 222, or administering a treatment to the patient 222. A treatment by module 268-B is sometimes called a second treatment, to distinguish it from first treatment 249.

Operation module 266 can perform its operation either at different capacities, such as a first capacity or at a second capacity. The first capacity could be full capacity, while the second capacity could be a lesser capacity, or none at all, in other words the operation is not performed at all. An example is now described.

Referring now to FIG. 2B, operation module 266 can perform its operation either at FULL CAPACITY, or at LESSER CAPACITY, according to an index 265. Index 265 can be implemented in a number of ways, such as in a software flag, an internal parameter value, and so on. In this example, full capacity is the first capacity.

Returning now to FIG. 2A, the operation of module 266 can be switched, as also reflected in the example of FIG. 2B. More specifically, when receiver 264 receives warning 245, a defensive action is taken, causing the operation of module 266 to be switched from a first capacity to where it can no longer be performed at the first capacity. In other words, it could now be performed either at a lesser capacity or not at all. The defensive action is reflected by arrow 267 in the example of FIG. 2B.

Moreover, the defensive action is later canceled, and module 266 can perform its operation again at the first capacity. The canceling action is reflected by arrow 277 in the example of FIG. 2B.

In some embodiments, from the warning, a nature of the impending first treatment is decoded. In some of those, the defensive action is neither taken nor canceled in response to the warning. This takes place if, for example, it is determined prospectively that the performance of the operation of module 266 can be repeated on the patient at the first capacity, with an effectiveness not affected by the first treatment.

Medical device 262 moreover optionally also includes an other module 270, for others of its operations, consistently with its use and function. For example, other module 270 could be a memory, a communications module, a user interface, and so on, as applicable. If other module 270 is a memory, it could store a record about the received warning. If medical device 262 is an external device, other module 270 could be a user interface, for communicating to a user of device 262 that warning 245 was received, information learned from warning 245, such as the nature and the impending duration of the first treatment, and so on. For example, there could be a message of the type “pacing about to start”, “shock about to be delivered”.

Medical device 262 may optionally include a controller 272 for controlling operation module 266, for other module 270 if provided, and perhaps for other internal operations.

These features are not limiting for device 262. Indeed, device 262 could also include features shown for device 241, and so on.

In some embodiments, warning 245 encodes an identifying code of the first medical device. This device code can be a device ID number, a serial number, a model or version, convey the configuration settings, and so on. In fact the nature of the first treatment can be implicit in the encoding of the device code.

In some embodiments, warning 245 encodes an identifying code of the patient. This patient code may have been learned by the first device in any number of ways, such as by being entered by a user of the first device, or being read automatically, by any number of readers. Such readers can be optical, RFID, and so on. The patient code may convey the patient name, patient ID, or other patient identifiers or characteristics that might have been previously encoded in the device. The patient code may be used for a second medical device, if attached to the patient, to distinguish who the impending first treatment is intended for.

The functions of this description may be implemented by one or more devices, such as controllers 252, 272, which include logic circuitry. A controller often includes a processor and a storage medium coupled with the processor. The storage medium has instructions stored thereon which, when executed by the processor, result in operation of the whole medical device. The device performs functions and/or methods as are described in this document. The logic circuitry may include a processor that may be programmable for a general purpose, or dedicated, such as microcontroller, a microprocessor, a Digital Signal Processor (DSP), etc. For example, the device may be a digital computer like device, such as a general-purpose computer selectively activated or reconfigured by a computer program stored in the computer. Alternately, the device may be implemented by an Application Specific Integrated Circuit (ASIC), and so on.

Moreover, methods are described below. The methods and algorithms presented herein are not necessarily inherently associated with any particular computer or other apparatus. Rather, various general-purpose machines may be used with programs in accordance with the teachings herein, or it may prove more convenient to construct more specialized apparatus to perform the required method steps. The required structure for a variety of these machines will become apparent from this description.

In all cases there should be borne in mind the distinction between methods in this description, and the method of operating a computing machine. This description relates both to methods in general, and also to steps for operating a computer and for processing electrical or other physical signals to generate other desired physical signals.

Programs are additionally included in this description, as are methods of operation of the programs. A program is generally defined as a group of steps leading to a desired result, due to their nature and their sequence. A program is usually advantageously implemented as a program for a computing machine, such as a general-purpose computer, a special purpose computer, a microprocessor, etc.

Storage media are additionally included in this description. Such media, individually or in combination with others, have stored thereon instructions of a program made according to the invention. A storage medium according to the invention is a computer-readable medium, such as a memory, and is read by the computing machine mentioned above.

Performing the steps or instructions of a program requires physical manipulations of physical quantities. Usually, though not necessarily, these quantities may be transferred, combined, compared, and otherwise manipulated or processed according to the instructions, and they may also be stored in a computer-readable medium. These quantities include, for example electrical, magnetic, and electromagnetic signals, and also states of matter that can be queried by such signals. It is convenient at times, principally for reasons of common usage, to refer to these quantities as bits, data bits, samples, values, symbols, characters, images, terms, numbers, or the like. It should be borne in mind, however, that all of these and similar terms are associated with the appropriate physical quantities, and that these terms are merely convenient labels applied to these physical quantities, individually or in groups.

This detailed description is presented largely in terms of flowcharts, display images, algorithms, and symbolic representations of operations of data bits within at least one computer readable medium, such as a memory. Indeed, such descriptions and representations are the type of convenient labels used by those skilled in programming and/or the data processing arts to effectively convey the substance of their work to others skilled in the art. A person skilled in the art of programming may use these descriptions to readily generate specific instructions for implementing a program according to the present invention.

Often, for the sake of convenience only, it is preferred to implement and describe a program as various interconnected distinct software modules or features, individually and collectively also known as software. This is not necessary, however, and there may be cases where modules are equivalently aggregated into a single program with unclear boundaries. In any event, the software modules or features of this description may be implemented by themselves, or in combination with others. Even though it is said that the program may be stored in a computer-readable medium, it should be clear to a person skilled in the art that it need not be a single memory, or even a single machine. Various portions, modules or features of it may reside in separate memories, or even separate machines. The separate machines may be connected directly, or through a network, such as a local area network (LAN), or a global network, such as the Internet.

It will be appreciated that some of these methods may include software steps that may be performed by different modules of an overall software architecture. For example, data forwarding in a router may be performed in a data plane, which consults a local routing table. Collection of performance data may also be performed in a data plane. The performance data may be processed in a control plane, which accordingly may update the local routing table, in addition to neighboring ones. A person skilled in the art will discern which step is best performed in which plane.

An economy is achieved in the present document in that a single set of flowcharts is used to describe both programs, and also methods. So, while flowcharts are described in terms of boxes, they can mean both method and programs.

For this description, the methods may be implemented by machine operations. In other words, embodiments of programs are made such that they perform methods of the invention that are described in this document. These may be optionally performed in conjunction with one or more human operators performing some, but not all of them. As per the above, the users need not be collocated with each other, but each only with a machine that houses a portion of the program. Alternately, some of these machines may operate automatically, without users and/or independently from each other.

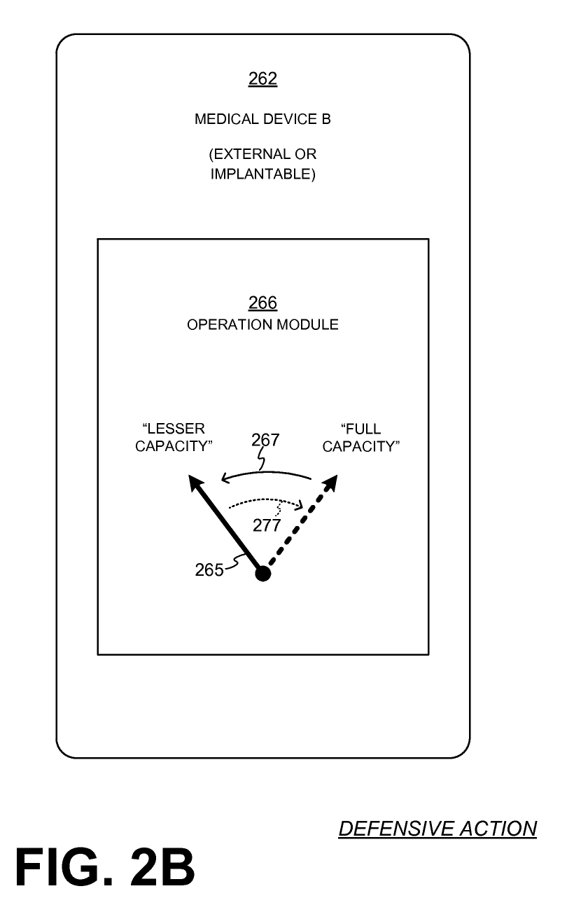

Methods according to the embodiments are now described, with reference to FIG. 3.

FIG. 3 shows a flowchart 340 for describing methods according to embodiments. These methods are for a first medical device to administer a first treatment to a patient, while a second medical device could be attached to the patient without such prior knowledge by the first medical device. The methods of flowchart 340 may also be practiced by first medical device 241.

According to an operation 345, a warning 245 is transmitted about an impending administration of the first treatment. Preferably warning 245 is suitable for receipt by the second medical device contemplated that could be attached to the patient. With reference to FIG. 3, warning 245 can be transmitted at a time T5 on a time axis.

In some embodiments, a preparatory operation for administering the first treatment is performed by the first medical device before the first treatment is administered. In these embodiments, the warning is transmitted responsive to the preparatory operation being performed.

According to optional operations, a record can be stored about the transmitted warning. Moreover, if it turns out that a second medical device is indeed attached to the patient, an acknowledgement could be received in response to the transmitted warning. The acknowledgement can be received in any number of ways, such as directly from the second medical device, or from an external network on behalf of the second medical device. Moreover, a confirmation can be transmitted in response to receiving the acknowledgement, and so on.

According to a next operation 349, the first treatment is administered. The treatment is in accordance with the nature of the first medical device. With reference to FIG. 3, the treatment can be administered at a time T9 on the time axis.

According to an optional next operation 355, an all-clear is transmitted, after the first treatment is administered. The all-clear can be transmitted similarly to warning 245. With reference to FIG. 3, the treatment can be administered at a time T15 on the time axis.

In advanced embodiments, warning 245 also encodes a nature of the first treatment, although that is not necessary for practicing the invention. This way, the listening second medical device can also know what the treatment will be, and react accordingly. Protocols according to the invention can create a unified system for how each type of treatment is to be encoded, and so on.

Optionally and preferably, warning 245 also encodes an intended timing of the impending administration of the first treatment. This can be performed in a number of ways. For example, the intended timing can include one or more aspects, such as a start timing, an end timing, and a duration of the impending administration of the first treatment. A detailed example is now described.

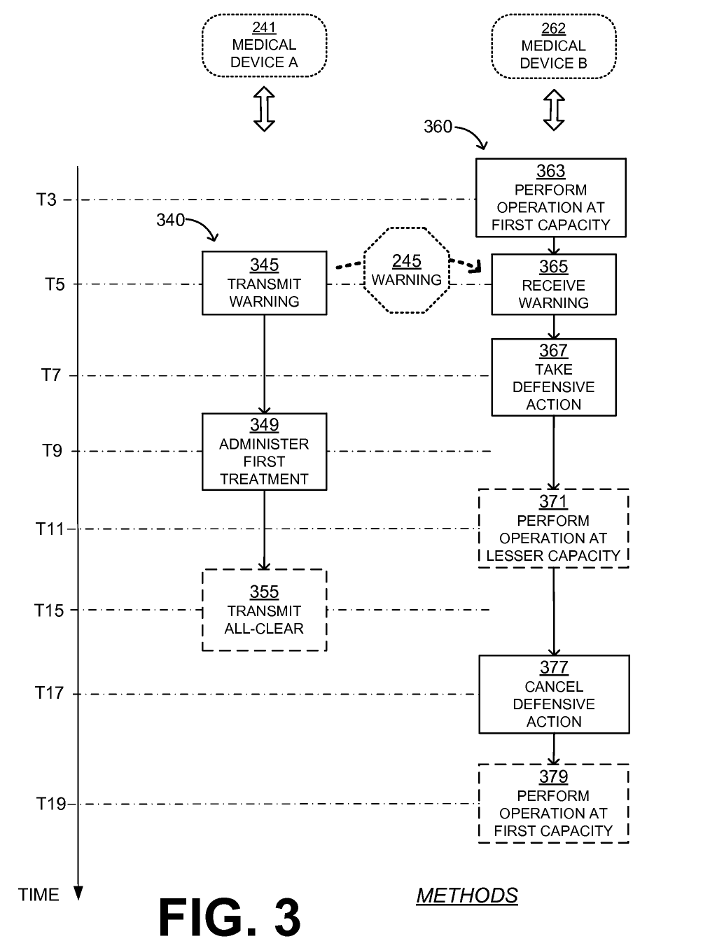

FIG. 4 shows a time axis with intercepts T5, T9 and T15, similar to those of FIG. 3. FIG. 4 also shows waveforms with features that correspond to intercepts T5, T9 and T15.

A waveform 444 is what might be output from warning module 244 of FIG. 2. Waveform 444 includes an optional preamble. The preamble can be for purposes of capturing the attention of a second medical device that could be listening. The preamble can also encode the nature of the first treatment.

After the preamble, waveform 444 includes a feature, shown as a pulse, which encodes the timing information for the impending administration of the first treatment. In other words, the timing information denotes one or more aspects about time T9, its start timing, end timing, duration, and so on.

Optionally, waveform 444 also includes an all-clear pulse, after the first treatment has been administered. The all-clear pulse will be especially useful for a second medical device that cannot interpret the timing information.

A waveform 449 is what might be output from treatment module 248 of FIG. 2A. In this example, the first treatment is an electrical biphasic pulse for stimulating an organ of the patient, occurring at a time T9. The timing of the pulse, its outward bounds, etc., can have been communicated by a waveform 444, reference to specific timing protocols, and so on.

In some embodiments, the first medical device might not know exactly when it will administer the first treatment, enough in advance to communicate a specific warning. In fact, the first medical device might not know whether it will administer the first treatment at all. In such embodiments the preamble might communicate the nature of the first treatment, and subsequent timing information pulses can relay the start timing of when the first treatment may be administered. Moreover, the preamble might further communicate additional information, such as the duration of the first treatment, if and when that is going to be delivered, and whether an all-clear should be expected, or implied after some communicated time.

Specifically with regards to the timing, in some embodiments the timing is communicated as a countdown. Moreover, in some embodiments the warning can include pulses. In such embodiments, an aspect of the timing is communicated as a distance between successive edges of the pulses. A detailed example is now described.

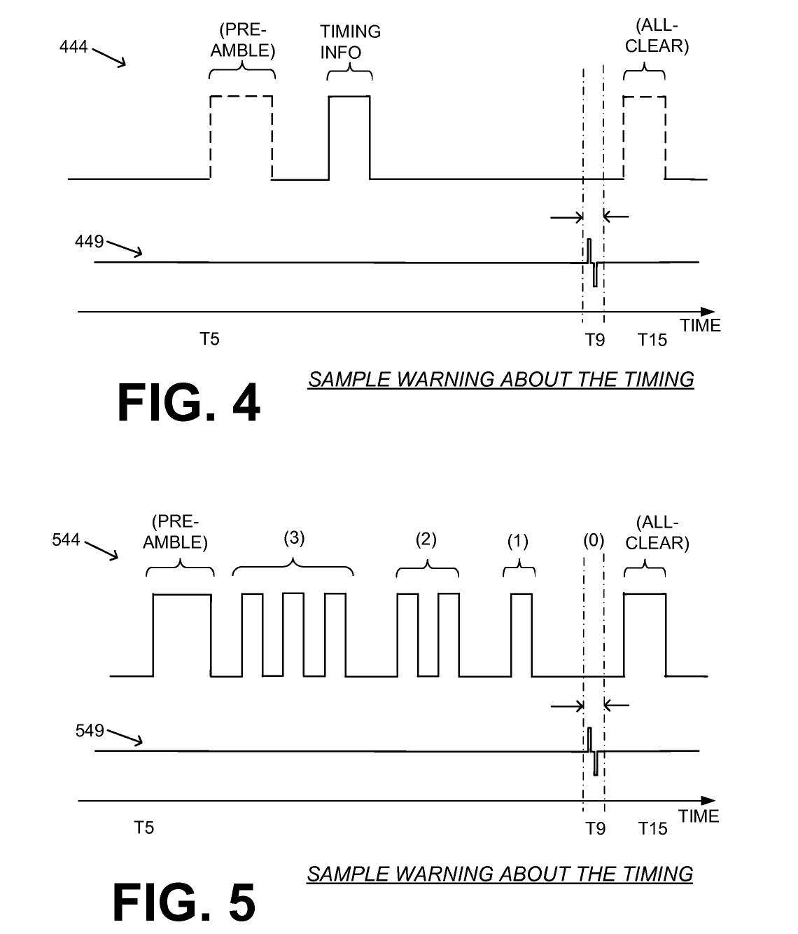

FIG. 5 shows a time axis with intercepts T5, T9 and T15, similar to those of FIG. 3. FIG. 5 also shows waveforms with features that correspond to intercepts T5, T9 and T15.

A waveform 544 is what might be output from warning module 244 of FIG. 2A. Waveform 544 includes a preamble similar to the preamble of FIG. 4. After the preamble, waveform 544 includes a series of pulses that perform a countdown (3, 2, 1) to time T9. Then an all-clear pulse is transmitted.

A waveform 549 is what might be output from warning module 244 of FIG. 2A, and in this example similar to waveform 449. Moreover, the duration of the countdown pulses has communicated the confines of the duration of the first treatment—in this case the first treatment happens within a time duration that is as long as one of the countdown pulses.

Returning to FIG. 3, a flowchart 360 is shown for describing additional methods according to embodiments. These methods are for a second medical device that could be attached to a patient. The methods of flowchart 360 may also be practiced by second medical device 262.

According to an operation 363, an operation of the second medical device is performed at a first capacity. The operation is either monitoring a physiological parameter of the patient, or administering a second treatment to the patient. With reference to FIG. 3, operation 363 can occur at a time T3 on the time axis.

According to a next operation 365, a warning is received about an impending administration of a first treatment to the patient. The first treatment would be by a first medical device, about which the second medical device has no prior knowledge. Operation 365 can occur at a time T5 on the time axis, concurrently with when warning 245 is transmitted per operation 345.

In some embodiments, from the warning, an identifying code of the first medical device is decoded. This device code can be stored in a memory, used to determine the nature of the impending first treatment, and so on.

In some embodiments, from the warning, an identifying code of the patient is decoded and stored in a memory. This can be used to determine whether the warning is for the same patient as the second medical device is attached to, and as to which the later-described defensive action is needed or whether one is needed at all. The received warning 245 can be stored. In addition, information about the received warning can be communicated to a user of the second medical device.

In some embodiments, an acknowledgement can be transmitted in response to the received warning. Moreover, a confirmation can be received in response to the transmitted acknowledgement.

According to a next operation 367, a defensive action is taken responsive to receiving the warning. The defensive action is such that the operation of the second medical device can no longer be performed at the first capacity. Operation 367 can occur at a time T7 on the time axis.

According to an optional next operation 371, the operation of the second medical device is indeed performed at a second capacity, which is lesser than the first capacity. Operation 371 can occur at a time T11 on the time axis.

According to a next operation 377, the defensive action is canceled. Accordingly, the operation of the second medical device can be performed again at the first capacity. Operation 377 can occur at a time T17 on the time axis. In some embodiments, an all-clear has been received at earlier time T15, and operation 377 is performed responsive to the received all-clear.

According to an optional next operation 379, operation 363 is repeated.

Another operation can be for the second medical device to initiate a self-checking function, after the defensive action is canceled. The self-checking function can be performed in any number of ways, and for any number of occasions. As one class of examples, the self-checking can be a device's routine internal self-testing. As another class of examples, the self-checking can be for testing for anything that might have changed as a result of the first treatment. Examples of such testing include, for example testing for lead integrity, if the first treatment is chest compression.

There are many possibilities of defensive actions according to embodiments. Of course, the appropriate defensive action or actions to a treatment are determined in connection with the nature of the first treatment, which is now expected to be administered by the device that transmitted the warning.

One intent of the defensive action is to preserve the operation of module 266 during the first treatment, and after it. For the afterwards operation, the intent is that module 266 will be able to operate just as effectively in the future, and at the same full capacity. In other words, for the afterwards operation, the defensive action is intended to prevent or reduce damage to components that is foreseeable from the treatment.

Another intent of the defensive action is to not subject the patient to inconsistent or redundant treatments. As such, one class of defensive actions is to make it so that second treatment module 268-B of operating module 266 cannot administer the second treatment that it is made for. This can be accomplished by disabling treatment module 268-B, whether as a circuit or in software, so that it will not operate.

In a broad class of embodiments, the first treatment involves electrical therapy, where an electrical impulse is discharged into a portion of the body. There are many possible defensive actions for this treatment.

In some embodiments, a defensive action is to increase the input impedance of sensors of the second medical device, sensors that could be measuring the ECG or other parameter. In some of these cases the sensors involve electrodes, as will be appreciated by a person skilled in the art.

In some embodiments, a defensive action is to increase the output impedance for ports that themselves can deliver electrical therapy. In some of these cases the sensors involve electrodes, as will be appreciated by a person skilled in the art.

In some embodiments, a defensive action is to make an internal adaptation so as to disregard sensor data received during the warned time window. This will prevent the accumulation of false data about the patient, as will be appreciated by a person skilled in the art.

Where the second medical device is an implantable device, an additional possible defensive action is to switch modes, as is described in US Patent Application Pub. No. US 2011/007706A1. One more possible action is to perform a controlled shutdown, with the further programming that the second medical device will reboot after some down time. The duration of the down time can be planned from information encoded in the received warning. Controlled shutdown is described in US Patent Application Pub. No. US2011/0238135, which is hereby incorporated by reference.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

The claimed invention is:

1. An external first medical device for administering a first treatment to a patient while a second medical device distinct from the first medical device could be attached to the patient without such knowledge by the first medical device, the external first medical device comprising:
a treatment module for administering the first treatment; and
a warning module for transmitting, before the first treatment is administered, a warning about the impending administration of the first treatment, the warning suitable for receipt by the second medical device.

2. The first device of claim 1, in which
the first device is one of an external defibrillator, a CPR chest compression device, a device that can inject drugs intravenously in a rescue scene, an ultrasound machine, a device for cooling the patient, an X-Ray machine, and an MRI machine.

3. The first device of claim 1, in which
the warning is transmitted through the body of the patient.

4. The first device of claim 1, in which
the warning is transmitted as a sound wave.

5. The first device of claim 1, in which
the warning is transmitted as a magnetic wave.

6. The first device of claim 1, in which
the warning is transmitted as an electromagnetic wave.

7. The first device of claim 1, in which
a preparatory operation for administering the first treatment is performed before the first treatment is administered, and
the warning is transmitted responsive to the preparatory operation being performed.

8. The first device of claim 1, further comprising:
a memory for storing a record about the transmitted warning.

9. The first device of claim 1,
in which the second medical device is indeed attached to the patient, and
further comprising: a receiving module for receiving an acknowledgement in response to the transmitted warning.

10. The first device of claim 9, in which
the acknowledgement is received directly from the second medical device.

11. The first device of claim 9, in which
the acknowledgement is received from an external network on behalf of the second medical device.

12. The first device of claim 9, in which
a confirmation is transmitted in response to receiving the acknowledgement.

13. The first device of claim 12, in which
the confirmation is transmitted by the warning module.

14. The first device of claim 12, in which
the confirmation is transmitted by an other module distinct from the warning module.

15. The first device of claim 1, in which
the warning module further transmits an all-clear after the first treatment is administered.

16. The first device of claim 1, in which
the warning encodes an identifying code of the first medical device.

17. The first device of claim 1, in which
the warning encodes an identifying code of the patient.

18. The first device of claim 1, in which
the warning encodes a nature of the first treatment.

19. The first device of claim 1, in which
the warning encodes an intended timing of the impending administration of the first treatment.

20. The first device of claim 19, in which
the timing includes one of a start timing, an end timing, and a duration of the impending administration of the first treatment.

21. The first device of claim 19, in which
the timing is communicated as a countdown.

22. The first device of claim 19, in which
the warning includes pulses, and
an aspect of the timing is communicated as a distance between successive edges of the pulses.

23. A controller for controlling an external first medical device to administer a first treatment to a patient while a second medical device distinct from the first medical device could be attached to the patient without such prior knowledge by the first medical device, the controller comprising: a processor and a storage medium coupled with the processor, the storage medium having instructions stored thereon which, when executed by the processor, result in:
transmitting a warning about an impending administration of the first treatment, the warning suitable for receipt by the second medical device; and
then administering the first treatment.

24. A method for an external first medical device to administer a first treatment to a patient while a second medical device distinct from the first medical device could be attached to the patient without such prior knowledge by the first medical device, the method comprising:
transmitting a warning about an impending administration of the first treatment, the warning suitable for receipt by the second medical device; and
then administering the first treatment.
